(19) United States
(12) Patent Application Publication
(10) Pub. No.: US 2026/0321379 A1
(43) Pub. Date: Oct. 15, 2026

(54) REAL-TIME ACTION-BASED PERSONALIZED CARE SYSTEM ADAPTABLE TO ANY PRESCRIPTION

(71) Applicant: MEDI.SOLA Co., Ltd., Seoul (KR)

(72) Inventors: Carl Jung Choon IM, Seoul (KR); Don Koo LEE, Yongin-si (KR); Seung Yeon LEE, Seongnam-si (KR)

(73) Assignee: MEDI.SOLA Co., Ltd., Seoul (KR)

(21) Appl. No.: 19/549,277

(22) Filed: Feb. 25, 2026

(30) Foreign Application Priority Data
Feb. 26, 2025 (KR) 10-2025-0025239

(51) Int. Cl. G16H 20/10; G16H 20/30; G16H 20/60; G16H 50/20; G16H 70/20; G16H 80/00

(52) U.S. Cl. CPC G16H 20/10; G16H 20/30; G16H 20/60; G16H 50/20; G16H 70/20; G16H 80/00

(57) ABSTRACT

The present invention relates to a real-time action-based healthcare system adaptable to any prescription, wherein a Lifestyle Medicine (LM) prescription is encoded as a Nutribiometric Script (NBS), an executable domain-specific language not restricted to predefined prescription formats or ontologies. Execution of the NBS dynamically instantiates a Lifestyle Medicine Space (LMS) as a computational evaluation environment in which evaluation variables, constraint relationships, and executable evaluation logic are defined and applied to user activities.

The system comprises: a user device configured to input user biometric measurements and user-specific data; a healthcare professional (HCP) device connected to the user device for reviewing, monitoring, and adjusting prescriptions; an online research publication search engine for accessing clinical research publications; a NutriBiometric (NB) Discovery module configured to interpret LM prescriptions provided by the user device or the HCP device, convert the prescriptions into executable NBS form, and derive lifestyle goals for chronic diseases, nutrition, and exercise; and a Lifestyle Medicine Activity (LMA) Discovery module configured to generate candidate executable activities to achieve the derived goals.

By executing the NBS, the system dynamically constructs the LMS without reliance on predefined measurement categories or ontologies, enabling evaluation of user activities through computational state representations within the LMS. The prescription processed by the NB server is executed as actionable lifestyle activities via the LMA Discovery module and an LMA server, allowing flexible adaptation to changing prescriptions while maintaining automated evaluation and tracking of lifestyle adherence.

### CROSS REFERENCE TO RELATED APPLICATION

This application claims priority to and the benefit of Korean Patent Application No. 10-2025-0025239, filed on Feb. 26, 2025, disclosures of which are incorporated herein by reference in its entirety.

### TECHNICAL FIELD

The present invention relates to a personalized real-time action-based healthcare system adaptable to any prescription. More specifically, the present invention relates to a personalized real-time action-based healthcare system adaptable to any prescription, capable of realizing flexible personalized care independent of prescription formats, schemas, or ontologies while operating on prescriptions. In particular, the present invention provides a computing architecture in which a prescription is encoded as an executable domain-specific program whose execution dynamically instantiates a computational evaluation environment at runtime, healthcare prescriptions representing one application domain of such architecture. In the present invention, executable script semantics define the structure of the computational evaluation environment itself, including evaluation dimensionality and dependency relationships, rather than merely supplying parameters to a pre-existing execution model. This is achieved by generating executable Lifestyle Medicine Activities (hereafter collectively referred to as LMAs) through machine execution of an executable domain-specific script, the generated LMAs being dynamically adjusted through runtime system execution and optionally presented via an application and web interface without altering executable evaluation logic, to enable real-time monitoring and computational evaluation of adherence to LMAs relative to patient-specific target constraint ranges defined within a Lifestyle Medicine Space (hereinafter collectively referred to as “LMS”).

The LMS is a dynamically instantiated computational evaluation environment in which evaluation dimensions, machine-readable evaluation variables, and constraint relationships are synthesized at runtime through execution of an executable domain-specific script, the LMS being structured around six core elements (hereinafter collectively referred to as “6 Pillars”) of Lifestyle Medicine: Nutrition, Physical Activity, Sleep, Stress Management, Social Connection, and Avoidance of Harmful Substances, for computational evaluation of adherence across lifestyle medicine domains. Based on target constraint ranges defined within the LMS, the system computes runtime-generated executable LMAs as executable outputs generated by executing an executable domain-specific script within the instantiated LMS (e.g., exercise, recipes, or diet selections), wherein such runtime-generated executable LMAs represent machine-generated action entities derived from computational evaluation of LMS state transitions relative to target constraint ranges.

### BACKGROUND ART

Existing computational systems lack mechanisms for representing lifestyle prescriptions as executable computational models capable of runtime evaluation.

As a result, although chronic diseases are on the rise in modern society and are often closely linked to ineffective lifestyle habits, and Lifestyle Medicine is emerging as a means to improve habits in daily lifestyles based on the 6 Pillars to promote health and prevent or manage chronic diseases and save the United States $75 billion annually, existing digital healthcare systems struggle to translate lifestyle medicine prescriptions into computationally actionable and dynamically adaptable interventions. Unlike conventional computational systems in which evaluation models, schemas, or dimensional structures are predefined and executable logic operates within such predefined structures, the present invention operates using executable prescriptions represented as domain-specific programs whose execution dynamically instantiates a computational evaluation environment.

In this architecture, evaluation dimensions, constraint relationships, and computational state representations may be generated as execution-time constructs derived from script semantics rather than from predefined system schemas or ontologies. Accordingly, the executable prescription does not merely define rules applied within an existing model but defines, through execution, the computational context in which evaluation occurs, enabling dynamic adaptation without restructuring underlying system architecture.

Such dynamic instantiation occurs without modification of underlying application code.

Accordingly, the absence of computational mechanisms capable of dynamically representing and executing lifestyle prescriptions gives rise to multiple technical and practical limitations in existing systems, including the following deficiencies:

First, existing LM guidelines generally provide only general information, and thus lack specific guidelines that can be practically applied to individual patients. That is, patients receive information about beneficial and harmful nutrients and habits, but are not provided with specific guidance on actionable LMAs tailored to each patient. For example, traditional lifestyle medicine recommendations such as “Increase your omega-3 intake” are given, but no specific, practical suggestions are provided, such as “Eat a handful of walnuts for breakfast.” This forces patients to design their own activities that fit their health goals and lifestyle habits, which can be a challenging task.

Second, LM prescriptions also need to be continuously adjusted as the patient's health status changes. However, existing systems fail to reflect changing patient conditions or new medical advancements and tend to rely on fixed prescriptions. For example, if a patient develops high blood pressure, they will need to switch to a new prescription system, which can lead to complex issues such as data migration problems or loss of existing health information.

Accordingly, there is a pressing need for a computing system configured to translate lifestyle prescriptions into executable intermediate representations through machine execution of an executable domain-specific script and to generate executable Lifestyle Medicine Activities (LMAs) as executable outputs that dynamically adapt to changing patient state without reliance on fixed prescription schemas.

The problems with these existing systems can be summarized as follows:

1. Limitations of general guidelines: Existing LM guidelines only provide patients with general information (e.g., “Increase your omega-3 intake”) and do not suggest specific, actionable activities (e.g., “Add 20 g of walnuts to your breakfast”). Patients must design their own activities to achieve their health goals, which can be a significant burden.

2. Existing computational systems do not provide mechanisms for representing lifestyle prescriptions as machine-executable domain-specific computational models capable of runtime modification within a dynamically instantiated computational evaluation environment. As a user's health state evolves, prescription constraints must be dynamically incorporated into the computational model without restructuring underlying system schemas. For example, when a diabetic patient develops hypertension, a prescription may require introduction of an additional constraint such as a sodium consumption cap, requiring the system to dynamically recompute evaluation dimensions and constraint relationships. However, conventional systems rely on predefined schema-defined evaluation variables and fixed dimensionality, such that when prescriptions evolve, complex technical problems arise, including data migration, loss of historical computational context, and inability to preserve consistent evaluation state representations across prescription updates.

Therefore, a system that can flexibly adapt to patients' changing health conditions as well as to new medical discoveries is needed. Additionally, it should be possible to generate runtime-generated executable LMAs as machine-generated runtime outputs derived from execution of an executable domain-specific script within a dynamically instantiated evaluation environment.

To address this, the present invention aims to provide a personalized, real-time, action-based healthcare system that can adapt to any prescription:

The present invention provides a flexible system that can handle all types of prescription protocols, including infinite variations of lifestyle medicine goals, utilizing NBS, thereby allowing for the simultaneous tracking of multiple nutritional goals. This system works by combining the NB Discovery module and the LMA Discovery module, where the NB Discovery module extracts relationships between chronic diseases and lifestyle goals (nutrients, exercise levels, and the like) from clinical studies, and the LMA Discovery module generates runtime-generated executable LMAs represented as executable activity scripts (LMAS), each configured for runtime projection into the LMS to produce computational state transitions relative to target constraint ranges.

In addition, the present invention proposes a multidimensional platform that can adapt to the patient's changing health status in real time and generate executable Lifestyle Medicine Activities (LMAs) as runtime-evaluated outputs within a dynamically instantiated computational environment. In particular, it tracks the patient's activities and goals in real time through a visual and multidimensional space called Lifestyle Medicine Space (LMS), and produces machine-generated executable outputs and evaluation state updates derived from runtime execution within the LMS.

Additionally, when a prescription is modified, the NBS is updated to reflect the change, and since it is an NBS-based system, no separate system migration is required. The LMA Discovery module can convert newly changed lifestyle goals into runtime-generated executable LMAs without modifying the source code. In addition, the NBS visualizes the patient's prescription adherence status and progress without modifying the system, providing effective feedback to both patients and healthcare professionals.

The technical architecture underlying the present invention is based on execution of an executable domain-specific script that dynamically instantiates a computational evaluation environment referred to herein as the Lifestyle Medicine Space (LMS).

The following are definitions of the key terms used herein:

Nutribiometrics (NB) refers to personalized daily Lifestyle Medicine (LM) objectives defined as evaluatable allowable intervals (constraint ranges) for improving a patient biometric state, wherein NB is represented as executable constraint ranges including representations encoded in an executable domain-specific script (NBS) and intermediate executable representations processed by a runtime execution engine implemented by the NCP Engine. In certain embodiments, NB represents a computational embodiment of a prescription expressed as executable constraint ranges suitable for runtime evaluation. For example, the prescription “Maintain 60-80 g of protein consumption daily” may be translated into a specific daily LM goal represented as NB for the purpose of increasing muscle mass. In this way, NB sets health improvement goals based on various biometric data such as the patient's weight, muscle mass, and blood sugar, and through this, it is possible to continuously monitor and improve the patient's health management and treatment outcomes. As used herein, the prefix “NB-” in a module name indicates that the corresponding module is functionally associated with NB representations, including generating, managing, storing, or processing such representations. This means that all these modules work based on the NB concept, which means not just a daily goal, but a NutriBiometrics system that includes the entire process of deriving executable NB representations from prescription inputs and managing such representations.

Nutribiometric Script (NBS) is a purpose-specific language designed to handle a variety of lifestyle medicine (LM) goals, without being restricted to specific prescription formats or ontologies.

Lifestyle Medicine Activity (LMA) refers to an executable activity entity generated through execution of an executable domain-specific script, the entity comprising:
executable behavioral parameters;
activity-to-LMS transformation logic defining a deterministic state-transition function; and
executable evaluation constraints interpretable by a runtime execution engine;
   wherein execution of the LMA by the runtime execution engine causes a computational state transition that updates a machine-maintained user state representation within a dynamically instantiated Lifestyle Medicine Space (LMS);
   wherein the LMA is not a recommendation, advisory output, or informational content but a machine-executable computational object whose execution semantics define state-transition operations within the LMS computational environment;
   and wherein execution of the LMA modifies a multidimensional evaluation state defined at runtime without modification of predefined system schemas, ontologies, or static evaluation models. Execution of an LMA updates computational state representations within the LMS regardless of whether any corresponding real-world behavior is performed, such that the LMA constitutes a machine-executable computational operator rather than a recommendation or informational output.

Lifestyle Medicine Activity Script (LMAS) is a purpose-specific language that encodes executable activity definitions and activity-to-LMS transformation logic, wherein the runtime execution engine implemented by the NCP Engine executes the LMAS to project candidate activities into the LMS and compute resulting computational state transitions.

The NB Discovery module serves to extract the relationship between chronic diseases and lifestyle goals (e.g., nutrient intake, exercise level) in clinical studies.

The LMA Discovery module generates executable Lifestyle Medicine Activities (LMAs) represented as executable activity scripts (LMAS) that are processed by a runtime execution engine implemented by the NCP Engine to produce computational state transitions toward target constraint ranges derived from the NB Discovery module.

Lifestyle Medicine Space (LMS) is a multidimensional computational evaluation environment instantiated by execution of an executable domain-specific script and configured to computationally evaluate user activities and target constraint ranges, wherein evaluation dimensions, machine-readable variables, and constraint relationships are dynamically generated at runtime based on execution of the executable domain-specific script rather than predetermined schemas or fixed ontologies. Within the LMS runtime environment, prescribed lifestyle goals are represented as executable constraint ranges (Green Zone) and user activities are projected as computational state representations whose evaluated positions are determined through runtime.

The evaluated state position produced by runtime execution computationally represents the difference between current user state and target constraint ranges, enabling automated real-time adherence determination. By visualizing these LMSs, it is possible to track the user's current location and whether or not they deviate from or adhere to the target range in real time. In addition, executable Lifestyle Medicine Activities can be evaluated through runtime simulation within the LMS to generate machine-evaluated state projections and runtime-generated executable LMAs representing alternative state transitions. For example, the Green Zone is a bar graphic that visually represents the prescription target, and it serves as a baseline for intuitively checking whether the user's activity is within the target range (for example, an LMA of “Maintain 60-80 g of protein daily” is represented as an executable constraint range within LMS. If the user's actual intake (e.g., 70 g) falls within the Green Zone, it means that the goal has been achieved).

Throughout this specification, the terms “user” and “patient” are used interchangeably depending on the context. This reflects that the present invention may be beneficial to both general users seeking to maintain their health and patients requiring chronic disease management, and either term is used to provide a more appropriate or powerful use case in each situation.

### SUMMARY OF THE INVENTION

### Technical Problem

Unlike conventional computational systems in which executable logic operates within predefined data models, schemas, or dimensional structures, the present invention provides a computing architecture in which a prescription is encoded as an executable domain-specific program whose execution dynamically instantiates the computational evaluation environment itself, such that evaluation dimensions, constraint relationships, and computational state representations are generated as execution-time constructs derived from program semantics rather than from predefined system models, thereby enabling schema-independent execution applicable to healthcare prescriptions and other domains.

In particular, the invention provides a prescription-defined virtual execution substrate in which computational topology, including evaluation dimensions, dependency relationships, constraint evaluators, and state vector structure, is deterministically generated from executable script semantics at runtime rather than predefined within application code or database schema.

Accordingly, building upon this execution architecture, an object of the present invention is to provide a computing system configured to translate Lifestyle Medicine prescriptions, including constraint conditions derived from clinical studies and medical research outputs, into executable intermediate computational representations and to generate executable Lifestyle Medicine Activities (LMAs) processed by a computational evaluation environment to address the above-mentioned problems. The primary technical challenge addressed by the present invention lies in transforming mathematical constraint conditions derived from clinical studies into executable constraint representations and runtime-evaluated activity outputs runtime-generated executable Lifestyle Medicine Activities (LMAs), wherein execution of each LMA produces computational state transitions within the Lifestyle Medicine Space (LMS). To address this, the present invention dynamically instantiates a multidimensional computational evaluation environment called the Lifestyle Medicine Space (LMS), within which executable constraint ranges derived from clinical studies and executable activity representations are computationally evaluated to determine user state relative to target constraints.

Furthermore, the present invention generates real-time, dynamically updated executable constraint ranges and runtime-generated executable LMAs derived from computational state transitions produced by runtime execution within the LMS, wherein user interfaces present evaluated computational state representations and machine-generated activity outputs supporting chronic disease management and prevention.

Consequently, the present invention transforms clinical research outputs into executable intermediate representations executed by a runtime execution engine implemented by the NCP Engine, thereby providing a personalized, real-time, action-based healthcare system adaptable to any prescription and enabling flexible, schema-independent personalized healthcare.

For example, if a specific clinical study recommends 60-80 g of protein intake per day, the executable constraint range is represented within the LMS as a Green Zone visualization representing a computational evaluation state, which is indicated by a green bar in FIG. 12. When a user consumes a pack of “The Protein”, that activity is represented by a dot on the LMS, indicating 60 g of protein intake. In this way, runtime evaluation engine implemented by NCP Engine performs computational evaluation within the LMS to determine whether the user's current state position relative to executable constraint ranges (Green Zone) represents compliance, deficiency, or excess, with visualization serving as a representation of the evaluated computational result.

### Solution to the Problem

A personalized real-time action-based healthcare system adaptable to any prescription according to the present invention comprises: a user device for inputting user biometric measurements and user-specific data; a healthcare professional (HCP) device connected to the user device for a healthcare professional to review, monitor, and adjust the prescription; an online research publication search engine for accessing clinical research publications via an online search engine; an NB (NutriBiometric) Discovery module configured to translate a Lifestyle Medicine (LM) prescription into an executable domain-specific script forming an intermediate executable representation processed by a runtime execution engine implemented by the NCP Engine, thereby deriving executable constraint ranges corresponding to lifestyle goals for chronic diseases, nutrients, and physical activity; and an LMA (Lifestyle Medicine Activity) Discovery module configured to generate executable Lifestyle Medicine Activities (LMAs) represented as executable activity scripts (LMAS) whose execution produces computational state transitions within a computational evaluation environment. The system is characterized in that executable domain-specific scripts derived from the prescription are executed by a runtime execution engine implemented by the NCP Engine to dynamically instantiate evaluation variables, constraint relationships, and runtime-generated executable LMAs without reliance on predefined measurement categories or fixed ontologies.

### Advantageous Effects of the Invention

As described above, the personalized real-time action-based healthcare system adaptable to any prescription according to the present invention has the advantage of improving patient adherence and health outcomes through automated computational evaluation and state-transition-based adherence determination. This is achieved by using NBS as an executable domain-specific script to generate dynamically updated executable constraint ranges and executable Lifestyle Medicine Activities (LMAs) evaluated within a computational evaluation environment, wherein interface outputs represent computational evaluation results guiding adherence.

Additionally, because the LMS is dynamically instantiated as a computational evaluation environment through execution of the executable domain-specific script, evaluation dimensions and constraint relationships are regenerated without manual system reconfiguration or migration when executable activities or prescriptions change.

Additionally, the system continuously converts clinical research outputs into executable intermediate representations processed by the runtime execution engine implemented by the NCP Engine, thereby generating updated runtime-generated executable LMAs and constraint parameters reflecting current clinical knowledge.

Additionally, runtime-generated executable Lifestyle Medicine Activities (LMAs) are presented through a user interface as machine-evaluated activity outputs, enabling users to incorporate healthy habits through system-generated executable action options.

Additionally, by promoting long-term adherence to LM goals, it has the advantage of significantly improving health outcomes for users managing chronic conditions.

### BRIEF DESCRIPTION OF DRAWINGS

FIG. 1 is a schematic overall configuration block diagram of a personalized real-time action-based healthcare system adaptable to any prescription according to the present invention.

FIG. 2 is a configuration block diagram of the user device of FIG. 1.

FIG. 3 is a configuration block diagram of the HCP device of FIG. 1.

FIG. 4 is a configuration block diagram of the NB Discovery module of FIG. 1.

FIG. 5 is a configuration block diagram of the NB extraction module of FIG. 4.

FIG. 6 is a configuration block diagram of the NB server of FIG. 1.

FIG. 7 is a configuration block diagram of the LMA Discovery module of FIG. 1.

FIG. 8 is a configuration block diagram of the LMA server of FIG. 1.

FIG. 9 is a configuration block diagram of the NCP engine of FIG. 1.

FIG. 10 is a configuration block diagram of the NCP simulator of FIG. 1.

FIG. 11 is a diagram illustrating an example where the NB Discovery module converts an online clinical research publication into an NBS. The generated NBS constitutes executable input to the runtime execution engine implemented by the NCP engine, which performs computational evaluation within the dynamically instantiated LMS.

FIGS. 12A and 12B are diagrams illustrating examples of a user interface of the user device.

FIG. 13 is a diagram illustrating the definition of NCP.

FIG. 14 is a diagram illustrating an example of an LMA list displayed on the user device.

FIG. 15 is a diagram illustrating an example of an LMA detailed list displayed on the user device.

FIG. 16 is a diagram illustrating an example of an NBS according to the present invention.

### DETAILED DESCRIPTION OF THE INVENTION

Hereinafter, a personalized real-time action-based healthcare system adaptable to any prescription according to the present invention will be described in detail through a detailed description of embodiments with reference to the drawings. In describing the present invention, if it is determined that a detailed description of a related known technology or configuration may unnecessarily obscure the gist of the present invention, the detailed description will be omitted. In addition, the terms described below are terms defined in consideration of the functions in the present invention, and may vary depending on the intention or custom of the client, operator, or user. Therefore, the definition should be made based on the contents throughout this specification.

In certain embodiments, the present invention implements a domain-specific language (DSL)-driven dynamic state-space instantiation engine configured to construct, at runtime, a computational evaluation environment in which evaluation dimensions, variables, and constraint relationships are defined by executable script expressions rather than predefined ontology schemas.

The underlying architecture operates as a general runtime constraint-state engine capable of modeling domain-specific processes as executable state-transition systems. While the present disclosure describes clinical nutrition as a primary embodiment, the runtime architecture is domain-agnostic and applicable to any prescription-defined evaluation domain.

While traditional clinical nutritional care programs rely on predefined assessment frameworks and manually defined intervention protocols implemented as human-managed workflows, the present invention computationally instantiates the nutritional care process as an executable state-transition system operating within a dynamically generated evaluation space, thereby enabling automated evaluation, adaptation, and extensibility beyond fixed clinical schemas.

In certain embodiments, the runtime execution engine operates as a programmable evaluation virtual machine configured to dynamically instantiate domain-specific state spaces and executable evaluation logic from prescription-derived executable DSL scripts interpreted by the runtime execution engine, rather than relying on predefined ontology schemas or fixed evaluation frameworks.

As used herein, the term “prescription” refers to an externally defined clinical, behavioral, or lifestyle objective or protocol. Upon processing by the system, such prescription is translated into executable constraint representations (“NB prescriptions”) and encoded as executable domain-specific scripts (e.g., Nutribiometric Scripts (NBS)), which are processed by the runtime execution engine to instantiate and operate within the Lifestyle Medicine Space (LMS).

The present invention is structured around the following four main functions:

1. Dynamic instantiation of the Lifestyle Medicine Space (LMS): Instantiating, at runtime, a computational evaluation environment (the LMS) by machine execution of an executable domain-specific script (e.g., Nutribiometric Scripts (NBS)) that dynamically generates machine-readable evaluation variables, evaluation dimensions, and constraint relationships without reliance on predefined measurement categories or ontologies. Unlike conventional systems that require predefined measurement schemas or fixed ontologies, execution of the domain-specific script dynamically defines evaluation dimensions at runtime, reducing system reconfiguration requirements and enabling extensible computational modeling without modification of underlying system architecture. The Lifestyle Medicine Space (LMS) is implemented as a machine-executable computational environment in which evaluation variables and constraint relationships are instantiated as executable state representations and evaluation structures processed by a computing device, thereby enabling automated computational evaluation that cannot be performed as a purely mental process. In certain embodiments, evaluation and state-transition operations within the dynamically instantiated Lifestyle Medicine Space require machine-executable processing of executable scripts, multidimensional state representations, and cumulative constraint evaluations that cannot be practically performed by a human mentally or with pen-and-paper analysis, thereby constituting a technical computational process rather than an abstract mental activity. In some embodiments, the dimensional structure of the Lifestyle Medicine Space (LMS) is instantiated directly from variables declared within the NBS expressions, without selecting from predefined ontology tables or fixed measurement category enumerations.

2. Definition of target constraint ranges as a Green Zone: Computing personalized, observable and measurable user goals as target constraint ranges within the dynamically instantiated LMS, and representing such target constraint ranges as a Green Zone associated with the six pillars of Lifestyle Medicine.

3. Automated evaluation of user activities via projection and state transitions: Transforming user-reported lifestyle activities into machine-readable activity representations and projecting each activity into the LMS to perform automated evaluation through computational state representations and state transitions, thereby determining in real time whether the user's current LMS position falls within, below, or above the target constraint ranges (Green Zone).

4. Generation of executable Lifestyle Medicine Activities (LMAs) to drive LMS state toward target constraints: Generating and managing specific user-tailored executable Lifestyle Medicine Activities (LMAs) whose projection into LMS produces computational state transitions within the LMS that maintain or move the user's LMS position toward the target constraint ranges (Green Zone). In certain embodiments, a Nutritional Care Process (NCP), including a clinical nutritional care program, is implemented as (i) an executable representation comprising a plurality of LMAs (e.g., LMAS-encoded activity definitions with parameters) and (ii) an evaluation objective expressed as NB prescription constraint ranges (Green Zone), wherein execution and evaluation of the LMAs by the runtime execution engine produce cumulative computational state transitions in the dynamically instantiated LMS that transform a current LMS state vector into a compliant LMS state vector within the Green Zone.

The above functional framework is realized through a runtime computational architecture described below:

### [Technical Implementation Architecture and Computational Model]

The foregoing functional framework is implemented as a computing-system architecture in which an executable domain-specific script is machine-executed to instantiate, at runtime, a Lifestyle Medicine Space (LMS) as a computational evaluation environment. In one embodiment, the present invention provides a computing-system architecture that transforms an unstructured Lifestyle Medicine (LM) prescription into an executable domain-specific script (e.g., an NBS) by translating the prescription into an intermediate executable representation interpreted by a runtime execution engine implemented by the NCP Engine, and by machine execution of the script instantiates at runtime a Lifestyle Medicine Space (LMS) as a computational evaluation environment, and executes the executable domain-specific script using a runtime execution engine implemented by the NCP Engine to update computational state representations within the LMS.

The NCP Engine comprises a runtime execution engine configured to interpret and execute executable domain-specific scripts, including Nutribiometric Scripts (NBS) and Lifestyle Medicine Activity Scripts (LMAS), and intermediate executable representations compiled therefrom, to perform evaluation scheduling, state management, and constraint processing, and to dynamically instantiate and update the Lifestyle Medicine Space (LMS) as a multidimensional computational evaluation environment, wherein higher-level functional operations are implemented as system modules invoked under control of the runtime execution engine. In this architecture, the runtime execution engine implemented by the NCP Engine operates as a centralized execution authority that deterministically controls interpretation, scheduling, and execution of executable domain-specific scripts and intermediate executable representations.

Accordingly, the runtime execution engine implements a prescription-defined virtual execution substrate in which computational topology, including evaluation dimensions, dependency relationships, constraint evaluators, and state vector structure, is deterministically generated from executable script semantics at runtime rather than predefined within application code or database schema.

The dynamically instantiated Lifestyle Medicine Space (LMS) therefore constitutes a runtime-generated execution environment defined by executable script semantics.

In some embodiments, the runtime execution engine and the dynamically instantiated Lifestyle Medicine Space (LMS) collectively operate as a virtual execution environment in which executable domain-specific scripts define executable evaluation logic analogous to programs executed within a virtual machine, such that evaluation dimensions, constraint relationships, and computational state representations are dynamically instantiated as execution-time constructs rather than predefined data structures. System modules, including the NB Discovery module and the LMA Discovery module, generate executable artifacts or candidate script structures, but execution semantics and computational evaluation are exclusively governed by the runtime execution engine. Accordingly, executable Lifestyle Medicine Activities (LMAs) are not independently generated recommendation outputs but are runtime-evaluated execution results produced through deterministic processing of executable scripts within the instantiated Lifestyle Medicine Space (LMS). In addition, in this architecture, Lifestyle Medicine Activity Scripts (LMAS) represent executable activity definitions encoded as domain-specific programs, while Lifestyle Medicine Activities (LMAs) represent runtime-evaluated activity instances produced through execution of LMAS by the runtime execution engine implemented by the NCP Engine.

Accordingly, the LMA Discovery module generates candidate LMAS defining executable activity logic, and the runtime execution engine executes the LMAS within the instantiated Lifestyle Medicine Space (LMS) to produce runtime-generated executable LMAs representing computational state transitions relative to target constraint ranges. In one embodiment, the disclosed computing-system architecture operates as a programmable execution substrate in which lifestyle medicine prescriptions encoded as executable domain-specific scripts are interpreted and executed by a runtime execution engine implemented by the NCP Engine. The runtime execution engine implemented by the NCP Engine performs script interpretation, evaluation scheduling, state management, and constraint processing, while dynamically instantiating the Lifestyle Medicine Space (LMS) as a computational evaluation environment. Unlike conventional systems based on fixed schemas, predefined workflows, or static ontologies, execution of the executable domain-specific script dynamically defines evaluation variables, constraint relationships, and evaluation dimensions at runtime, thereby generating machine-readable computational state representations within the Lifestyle Medicine Space (LMS). User-reported activities are transformed into machine-readable activity representations and projected into the LMS, where automated evaluation is performed by computing computational state transitions that update a user's current LMS position relative to target constraint ranges (Green Zone). The resulting computational state representations are stored and communicated among system modules to automatically control subsequent activity generation, scoring, monitoring, and interface outputs, thereby providing a concrete improvement in computer functionality by enabling schema-independent runtime evaluation and extensible computational modeling without reconfiguration of underlying system data structures.

The personalized real-time action-based healthcare system adaptable to any prescription according to the present invention is implemented as a runtime computational execution environment in which an executable domain-specific script (e.g., Nutribiometric Script (NBS)) is interpreted by system processors to instantiate the Lifestyle Medicine Space (LMS) as a machine-executable evaluation environment.

Execution of the executable domain-specific script dynamically defines evaluation dimensions, machine-readable variables, and constraint relationships that form computational state representations within the LMS.

In some embodiments, the NB Discovery Module optionally uses a Large Language Model (LLM) as an input-processing component to convert unstructured clinical research publications into executable domain-specific scripts used by the runtime execution engine implemented by the NCP Engine. The LLM, when used, functions solely as an optional input-to-script conversion component; runtime instantiation of evaluation dimensions, execution semantics, and computational state transitions are performed independently by the runtime execution engine implemented by the NCP Engine.

Executable Lifestyle Medicine Activities (LMAs), defined by Lifestyle Medicine Activity Scripts (LMAS), are projected into the LMS to produce computational state transitions representing changes in user position relative to personalized target constraint ranges. The LMS is instantiated at runtime based on evaluation variables defined in the executable domain-specific script rather than predetermined schemas.

### Runtime Execution Model and Intermediate Representation Architecture

In some embodiments, an executable domain-specific script, such as a Nutribiometric Script (NBS) or a Lifestyle Medicine Activity Script (LMAS), is processed through a compiler-runtime execution architecture configured to enable dynamic evaluation and execution.

### Compilation Stage

In some embodiments, the system includes a compilation stage configured to parse the executable domain-specific script and translate the parsed script into an intermediate executable representation (IR).

The IR defines one or more of:
evaluation variables representing computable evaluation dimensions;
constraint ranges defining allowable value intervals associated with the evaluation variables;
evaluation relationships defining logical, mathematical, or rule-based dependencies among the evaluation variables; and
executable evaluators configured to be executed during runtime.

The IR is generated independently of any predefined schema, ontology, or static data model.

### Intermediate Representation Structure

In some embodiments, the intermediate executable representation comprises:
a symbol table defining dynamically generated evaluation dimensions corresponding to symbols declared in the executable domain-specific script;
executable constraint definitions forming constraint ranges to be evaluated during runtime execution;
dependency relationships defining a computational evaluation graph specifying evaluation order and data dependencies among evaluation dimensions; and
executable evaluation logic interpretable by a runtime execution engine implemented by an NCP Engine.

The IR is self-contained such that the evaluation logic required for runtime execution is fully defined within the IR.

### Runtime Instantiation and Schema Synthesis

In some embodiments, the runtime execution engine implemented by the NCP Engine is configured to execute the IR to instantiate a Lifestyle Medicine Space (LMS) as a multidimensional computational evaluation environment.

Unlike systems that rely on predefined schemas or ontologies, evaluation dimensions within the LMS are synthesized at runtime based on symbols defined in the IR, such that:
evaluation dimensions are not required to be known at system design time; and
no structural modification of a database or storage schema is required to support newly defined prescriptions.

Accordingly, the LMS is dynamically constructed as a computational evaluation space whose structure is determined by the executable IR.

### State Machine Execution Model

In some embodiments, the LMS operates according to a computational state machine model.

In this model:
a state vector represents a current user position within the LMS;
runtime-generated executable Lifestyle Medicine Activities (LMAs) are applied as inputs to the state machine and trigger state transitions; and
constraint ranges defined in the IR operate as invariants evaluated against the state vector during execution.

State transitions are performed by applying the executable evaluators defined in the IR to the state vector.

### Execution Trace and Reproducibility

In some embodiments, the runtime execution engine is further configured to generate execution traces.

Each execution trace links:
an identifier or version of the IR,
an input activity representation corresponding to an executed LMA, and
a resulting computational state of the LMS.

The execution traces enable reproducible evaluation and computational provenance independent of clinical interpretation.

Like reference numerals refer to like elements throughout the drawings.

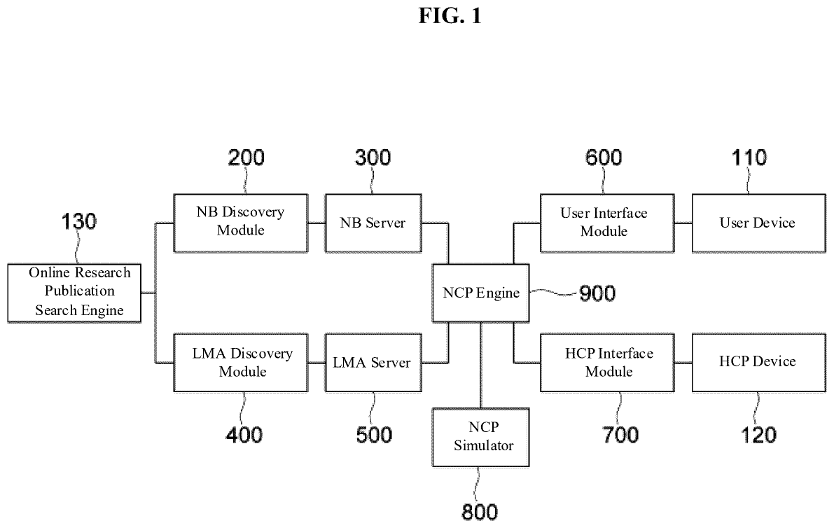

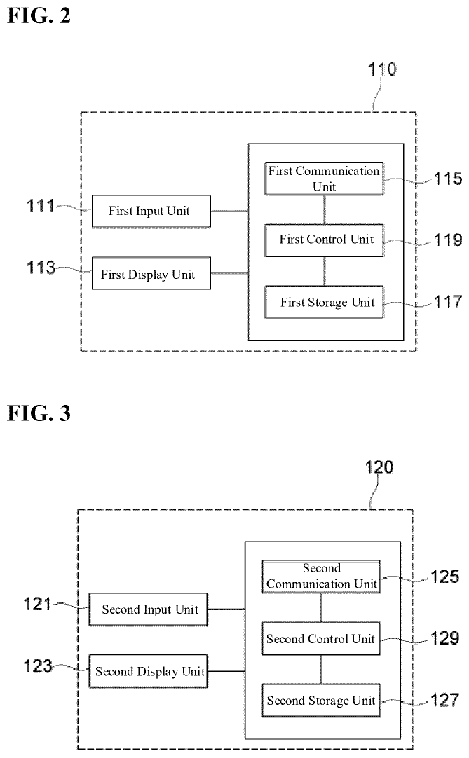

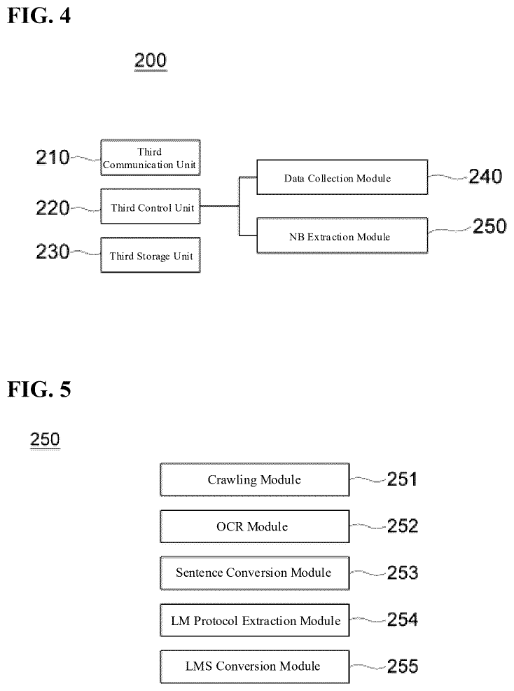

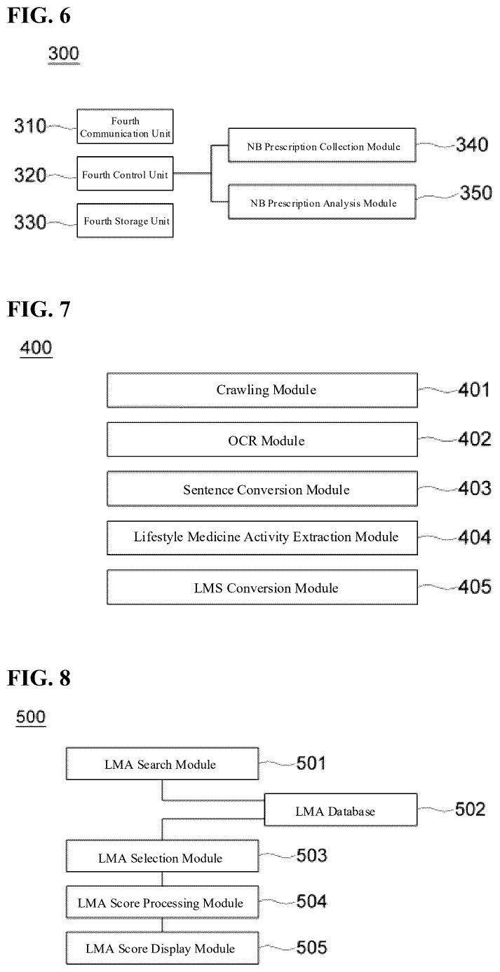

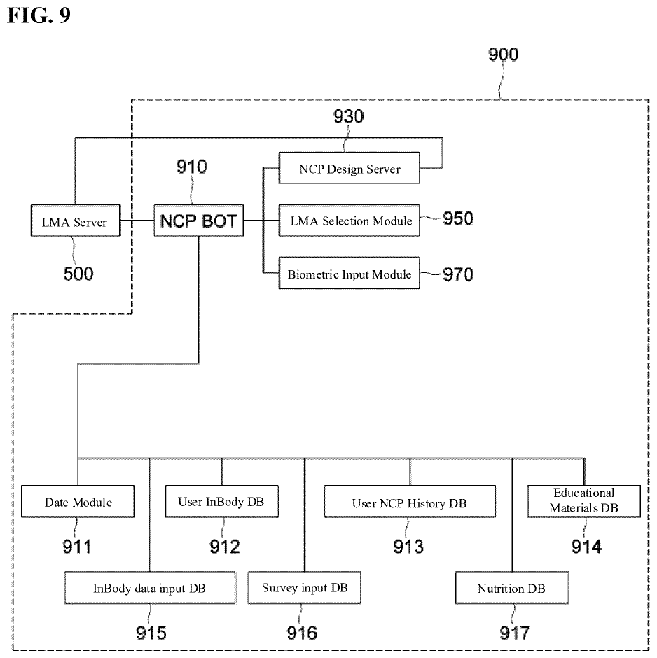

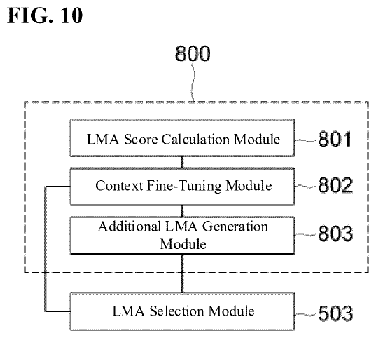

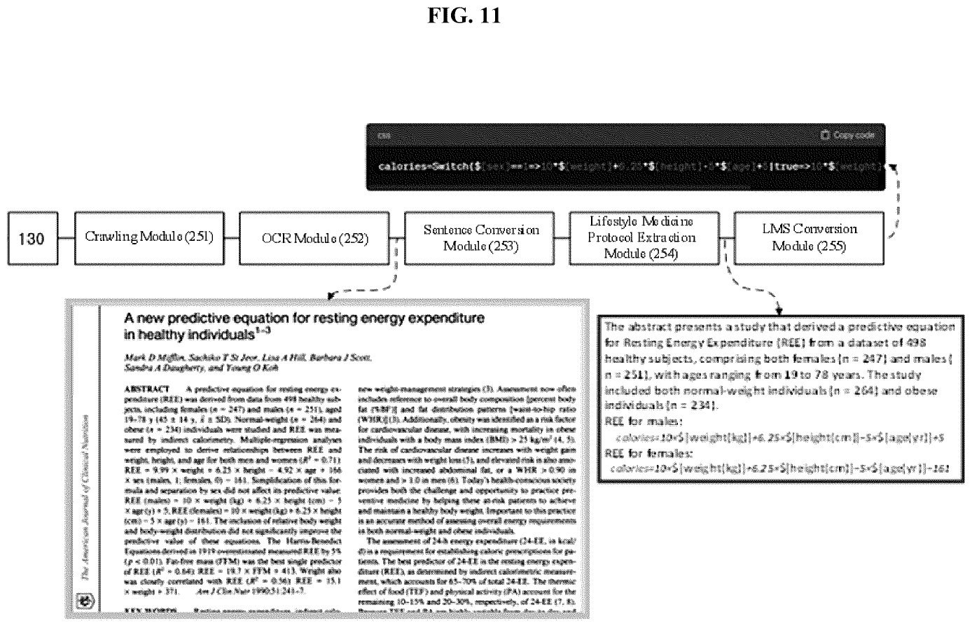

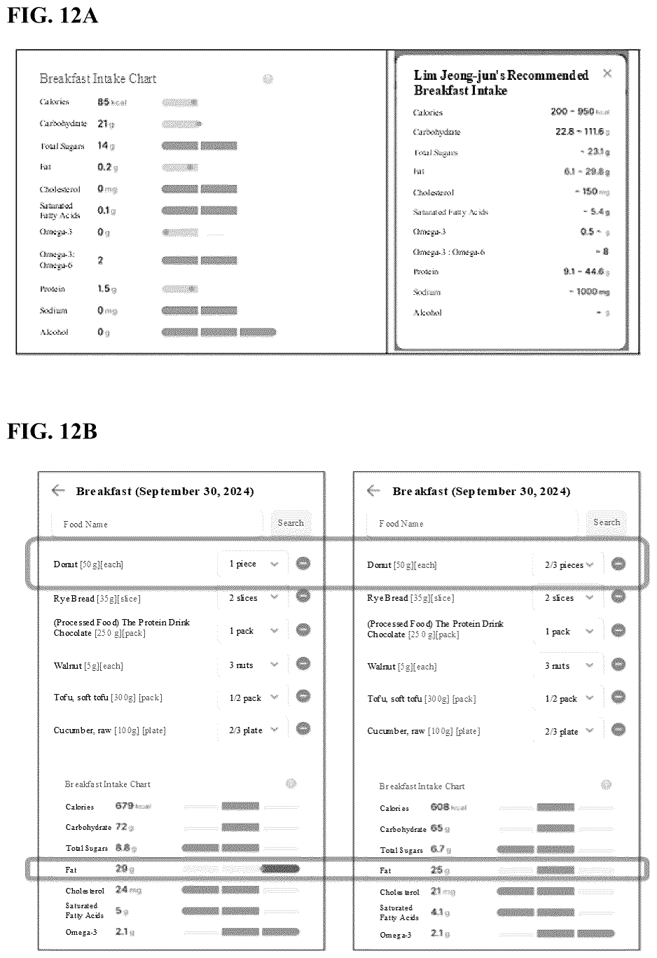

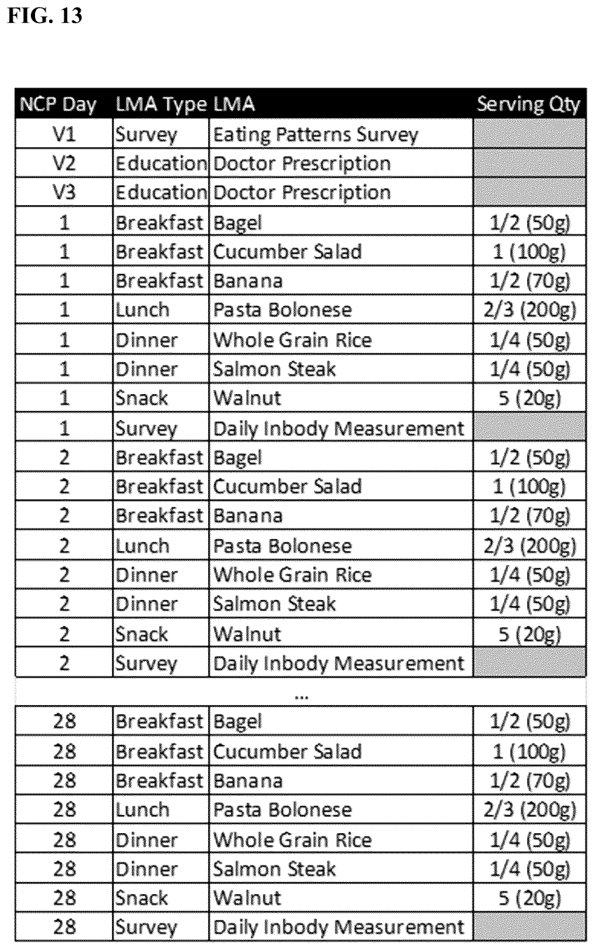

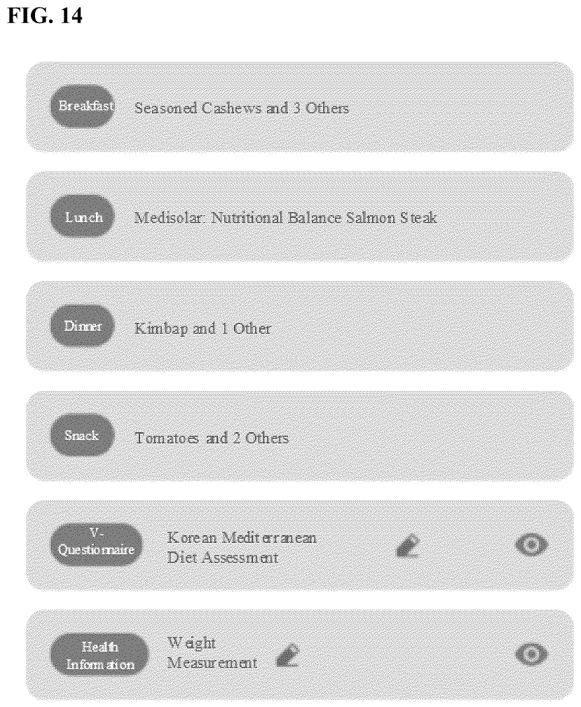

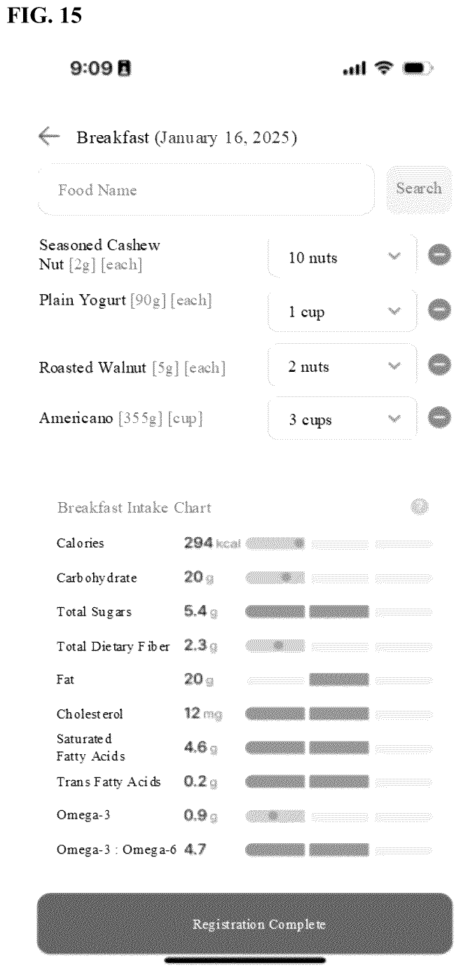

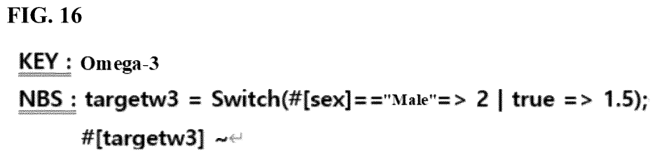

FIG. 1 is a schematic overall configuration block diagram of a personalized real-time action-based healthcare system adaptable to any prescription according to the present invention; FIG. 2 is a configuration block diagram of the user device of FIG. 1; FIG. 3 is a configuration block diagram of the HCP device of FIG. 1; FIG. 4 is a configuration block diagram of an NB Discovery module according to embodiments of the present invention; FIG. 5 is a configuration block diagram of the NB extraction module of FIG. 4; FIG. 6 is a configuration block diagram of the NB server of FIG. 1; FIG. 7 is a configuration block diagram of the LMA Discovery module of FIG. 1; FIG. 8 is a configuration block diagram of the LMA server of FIG. 1; FIG. 9 is a configuration block diagram of the NCP engine of FIG. 1; FIG. 10 is a configuration block diagram of the NCP simulator of FIG. 1; FIG. 11 is a diagram illustrating an example where the NB Discovery module converts an online clinical research publication into an NBS; FIGS. 12A and 12B are diagrams illustrating examples of a user interface of the user device; FIG. 13 is a diagram illustrating the definition of NCP; FIG. 14 is a diagram illustrating an example of an LMA list displayed on the user device; FIG. 15 is a diagram illustrating an example of an LMA detailed list displayed on the user device; and FIG. 16 is a diagram illustrating an example of an NBS according to the present invention.

Now, with reference to FIG. 1, we will examine in detail a personalized real-time action-based healthcare system adaptable to any prescription according to the present invention.

As illustrated in FIG. 1, the personalized real-time action-based healthcare system adaptable to any prescription according to the present invention includes a user device 110, an HCP (Healthcare Professional) device 120, an online research publication search engine 130, an NB Discovery module 200, an NB server 300, an LMA Discovery module 400, an LMA server 500, a user interface module 600, an HCP interface module 700, an NCP (Nutritional Care Process) simulator 800, and an NCP engine 900.

Here, the user device 110 is connected to the HCP device 120 via a communication network (not shown), and the HCP device 120 is connected to the user device 110, the online research publication search engine 130, the NB Discovery module 200, the NB server 300, the LMA Discovery module 400, the LMA server 500, the user interface module 600, the HCP interface module 700, the NCP simulator 800, and the NCP engine 900 via the communication network.

Additionally, the user device 110 is used to input user-specific data, such as biometric measurements including the user's height, weight, and blood sugar levels, and food intake data (e.g., name and amount of food consumed). The types of biometric measurements collected as part of the user-specific prescription are flexible and not limited to predefined categories or ontologies.

This flexibility is achieved through the integration of a purpose-specific language called NBS, which allows for describing generic prescriptions without relying on fixed measurement categories, thereby ensuring personalized treatment adaptable to a variety of biometric inputs and lifestyle activities.

In addition, the user device 110 may be implemented as an electronic device capable of performing communication, receiving user input, and outputting a screen, or a similar device, such as, for example, a smart phone, a portable terminal, a mobile terminal, a personal digital assistant (PDA), a portable multimedia player (PMP), a telematics device, a navigation device, a personal computer, a notebook computer, a slate PC, a tablet PC, an ultrabook, a wearable device, a WiBro device, an Internet Protocol Television (IPTV) device, a smart TV, a digital broadcasting device, an audio video navigation (AVN) device, an audio/video (A/V) system, a flexible terminal, and the like.

Additionally, the HCP device 120 allows healthcare providers to interact with the system for professional purposes. Like the user device 110, the HCP device 120 can collect and display user data, but is tailored to enable healthcare professionals to review, monitor, and adjust prescriptions.

Such an HCP device 120 can be implemented as various electronic devices, such as, for example, a smart phone, a portable terminal, a mobile terminal, a personal digital assistant (PDA), a PMP, a telematics device, a navigation device, a personal computer, a notebook computer, a slate PC, a tablet PC, an ultrabook, a wearable device, a WiBro device, an IPTV device, a smart TV, a digital broadcasting device, an AVN device, an A/V system, a flexible terminal, and the like. Healthcare providers can securely access the system to make specific adjustments for users and monitor lifestyle adherence.

Additionally, the online research publication search engine 130 provides access to clinical research publications through online search engines such as Google, Yahoo, Bing, Naver, Daum, and the like. The online research publication search engine 130 provides machine-accessible clinical research inputs related to the 6 Pillars of Lifestyle Medicine and chronic diseases via a communication network. This connection supplies research-derived constraints and parameters that are compiled into an intermediate executable representation and executed by a runtime execution engine implemented by the NCP Engine to update executable constraint ranges and to generate executable Lifestyle Medicine Activities (LMAs) for presentation via the user interface.

In addition, the communication network may be implemented using at least some of the communication methods developed in the past, present, or to be made available in the future, such as LTE (Long Term Evolution), LTE-A (LTE-Advanced), Wi-Fi, LAN (Local Area Network), WAN (Wide Area Network), CDMA (Code Division Multiple Access), TDMA (Time Division Multiple Access), WiBro (Wireless Broadband), GSM (Global System for Mobile Communications), and the like. Hereinafter, for convenience, the communication network will not be mentioned, and the description will proceed as if the user device 110, HCP device 120, online research publication search engine 130, NB Discovery module 200, NB server 300, LMA Discovery module 400, LMA server 500, user interface module 600, HCP interface module 700, NCP simulator 800, and NCP engine 900 communicate directly.

Additionally, the NB Discovery module 200 serves to interpret and analyze the LM prescription provided by the user device 110 or the HCP device 120. The NB Discovery module 200 uses NBS to convert the prescription into a format understandable by the system, ensuring that it is not limited to predefined measurement categories or ontologies, and transmits the analyzed prescription to the NB server 300 for further processing.

Furthermore, the NB server 300 processes data received from the NB Discovery module 200 and organizes derived lifestyle goals into executable constraint parameters forming part of an intermediate executable representation configured for execution by a runtime execution engine implemented by the NCP Engine. The NB server 300 communicates with the LMA Discovery module 400 and the LMA server 500 to provide prepared prescription data, constraint parameters, and supporting metadata used during runtime evaluation. In some embodiments, the NB server 300 manages mapping of user activities to the six pillars of Lifestyle Medicine and maintains structured representations of personalized target constraint ranges; however, authoritative computational evaluation, including LMS state transitions, Green Zone compliance determination, and execution of domain-specific scripts (e.g., NBS), is performed by the runtime execution engine implemented by the NCP Engine.

In addition, the LMA Discovery module 400 serves to analyze and generate an LMA based on the user's current health status, prescription, and input data, calculating user-tailored lifestyle behaviors such as specific dietary adjustments or physical activities based on the prescription processed by the NB server 300.

Additionally, the LMA server 500 cooperates with the LMA Discovery module 400 to store, index, and manage executable Lifestyle Medicine Activities (LMAs) and associated metadata. The LMA server 500 maintains records of user-selected and completed activities and provides activity data to other system components for runtime evaluation. In some embodiments, the LMA server 500 tracks user activity history and facilitates retrieval of LMA information for display via user interface components; however, authoritative computational evaluation, including determination of whether user activities satisfy target constraint ranges (Green Zone) and calculation of LMS state transitions, is performed by the runtime execution engine implemented by the NCP Engine.

Furthermore, the user interface module 600 provides a user interaction platform via the user device 110, displays outputs derived from runtime evaluation results within the LMS, collects user input, updates progress toward lifestyle goals, and seamlessly integrates with the LMA LMA Discovery module 400 to provide real-time feedback based on user input.

Also, the HCP interface module 700 allows healthcare providers to interact with the system via the HCP device 120, provides healthcare professionals with secure access to user data, enables adjustment of prescriptions, review of user activities, and tracking of compliance, all of which occur within the framework of the Nutritional Care Process (NCP) architecture.

Additionally, the NCP simulator 800 acts as a computational engine that simulates the impact of the LMA on user health, modeling computational state transitions representing changes in user position within the Lifestyle Medicine Space (LMS) associated with the six pillars of Lifestyle Medicine based on inputs from the LMA Discovery module 400 and the NB server 300, thereby generating predictions regarding future compliance and health outcomes. The simulation produces updated computational state representations within the Lifestyle Medicine Space (LMS), which are stored, transmitted, and used by system modules to automatically control subsequent activity generation, evaluation scoring, and interface outputs.

Furthermore, the NCP engine 900 implements the runtime execution engine responsible for executing executable domain-specific scripts and coordinating higher-level system modules through runtime evaluation processing, calculating personalized health goals for each user and dynamically adjusting them in real time according to user input, changing prescriptions, or evolving health conditions. This NCP engine 900 communicates directly with the LMA Discovery module 400 and the LMA server 500 to continuously improve the user's LMA.

The personalized real-time action-based healthcare system adaptable to any prescription according to the present invention as described above is connected via a communication network including the user device 110, HCP device 120, online research publication search engine 130, and backend components. This communication network enables real-time data exchange, allowing the system to process and respond to user input, adjustments by healthcare providers, and research updates in a clinical database (DB) accessed via the online research publication search engine 130, and ensures seamless connectivity between all system components using the various communication protocols described above.

Now, with reference to FIG. 2, the user device according to the present invention will be examined in detail.

As illustrated in FIG. 2, the user device 110 may include a first input unit 111, a first display unit 113, a first communication unit 115, a first storage unit 117, and a first control unit 119.

Here, the first input unit 111 converts user actions into input signals and transmits them to the first control unit 119, supporting devices such as a keyboard, mouse, touchscreen, touchpad, keypad, or voice input to allow the user to interact with the system in various ways. This first input unit 111 processes information requests and transmits the processed information requests to the first control unit 119 for further processing.

Additionally, the first display unit 113 outputs a screen under the control of the first control unit 119. This first display unit 113 displays user-specific information, such as prescribed nutrient ranges (minimum and maximum values) and actual nutrient intake based on user input. The first display unit 113 operates in conjunction with the user interface module 600, which retrieves a NutriBiometric Prescription (NBP) from the NB server 300 and receives actual LMA records from the LMA server 500. Here, the user interface module 600 provides relevant feedback, and the provided relevant feedback is displayed on the first display unit 113.

In addition, the first display unit 113 may be implemented as, for example, a Liquid Crystal Display (LCD), a Light Emitting Diode (LED), an Organic Light Emitting Diode (OLED), a projector, or other display devices available in the present, past, or future. In some cases, a component using other methods capable of conveying information to the customer, such as voice output or vibration, may be used instead of the first display unit 113, rather than screen output.

Additionally, the first communication unit 115 manages data exchange between the user device 110 and external systems such as the user interface module 600. It transmits user-generated data, such as biometric measurements and recorded activities, to the system server for analysis and retrieves runtime-generated executable LMAs and evaluated state outputs.

Furthermore, the first storage unit 117 serves to store user data, inputs, local processing results, and cached data from external servers, and can process or transmit the stored data to provide it to the first control unit 119 when necessary.

Also, the first control unit 119 coordinates all operations of the user device 110, manages input, output, communication, and storage, processes data from the first input unit 111, sends analysis requests to the user interface module 600, and controls information to be displayed on the first display unit 113 according to data received from the NB server 300 and the LMA server 500. The first control unit 119 manages data transmission via the first communication unit 115 to provide real-time feedback and dynamically adjust displayed runtime-generated interface outputs, based on the most up-to-date user data and computational state representations updated by execution of the intermediate executable representation in the runtime execution engine implemented by the NCP Engine.

Meanwhile, the user device 110 can process various types of inputs, such as touch, voice, and image inputs, interpret user interactions (e.g., taps, swipes), and display related information such as graphic data, audio signals, or vibrations. For example, if a user records food intake, the first display unit 113 may display nutritional consumption details based on information retrieved from the NB server 300 and the LMA server 500.

Additionally, the first control unit 119 and the first storage unit 117 may be implemented as individual devices or distributed across multiple devices depending on the system architecture; while described as single entities for simplicity, communication between components may be required in a distributed implementation. Data exchange between the user device 110 and external systems may be controlled by the first communication unit 115 under the supervision of the first control unit 119, or directly managed by the first control unit 119.

Now, with reference to FIG. 3, the HCP device according to the present invention will be examined in detail.

As illustrated in FIG. 3, the HCP device 120 may include a second input unit 121, a second display unit 123, a second communication unit 125, a second storage unit 127, and a second control unit 129.

Here, the second input unit 121, the second display unit 123, the second communication unit 125, the second storage unit 127, and the second control unit 129 are similar to the first input unit 111, the first display unit 113, the first communication unit 115, the first storage unit 117, and the first control unit 119 of FIG. 2, but will be examined in more detail as follows.

First, the second input unit 121 converts the healthcare provider's actions into input signals and transmits these signals to the second control unit 129. Like the first input unit 111, it supports various input devices such as a keyboard, mouse, touchscreen, touchpad, keypad, or voice input system. This second input unit 121 processes the healthcare provider's information request and transmits it to the second control unit 129.

Additionally, the second display unit 123 provides visual or non-visual feedback to the healthcare provider and displays information about user LMA and prescriptions, including prescribed nutritional ranges, such as minimum and maximum values, and actual consumption data. This second display unit 123 may be implemented using various display technologies, such as LCD, LED, OLED, and projectors, and can provide alternative feedback, such as audio prompts or vibrations, to alert the healthcare provider.

Furthermore, the second communication unit 125 handles data exchange between the HCP device 120 and external components such as the HCP interface module 700. It transmits data, such as user activity records and prescription updates, to the system server for further processing and retrieves relevant analyses and runtime-generated executable LMAs for the healthcare provider.

Also, the second storage unit 127 stores data related to the healthcare provider's system interaction.

The stored data includes local processing results, user data, and information cached from external sources. The second storage unit 127 processes or transmits data to provide it to the second control unit 129 as needed.

In addition, the second control unit 129 supervises the overall functions of the HCP device 120, coordinates inputs from the second input unit 121, manages data storage and retrieval in the second storage unit 127, and controls the second communication unit 125 to exchange data with external modules.

Furthermore, the second control unit 129 processes inputs from the healthcare provider, retrieves necessary data from external servers such as the NB server 300 and the LMA server 500, and determines the appropriate output to be displayed on the second display unit 123.

Meanwhile, the HCP device 120, like the user device 110, can detect various types of inputs, such as touch, voice, and image or video inputs, and can identify when a selection has been made, moved, or completed. For example, a sustained touch, a double tap, or a movement of the input location may indicate an action taken by the healthcare provider.

Additionally, the second display unit 123 can display minimum and maximum prescribed nutrient consumption values and actual consumption levels for a specific user. This information is displayed when the HCP device 120 requests data from the HCP interface module 700. The HCP interface module 700 requests the NB server 300 to retrieve a NutriBiometric (NB) prescription, which includes nutrient consumption-related runtime-generated executable LMAs based on the user's evaluation date and user ID.

Also, the HCP interface module 700 requests LMA records from the LMA server 500. After the requested LMA records are processed, the HCP interface module 700 collects information from both the NB server 300 and the LMA server 500, transmits it back to the HCP device 120, and displays it on the second display unit 123.

Now, with reference to FIG. 4, the NB Discovery module according to the present invention will be examined in detail.

As illustrated in FIG. 4, the NB Discovery module 200 includes a third communication unit 210, a third control unit 220, and a third storage unit 230. The NB Discovery module 200 serves to extract and convert relevant protocols into executable domain-specific scripts (NBS) used by the runtime execution engine implemented by the NCP Engine and convert the extracted protocols into NBS for nutritional protocols and other LMAs. The NB Discovery module 200 searches for new research papers in online publications, extracts formulas from these research papers, and integrates them into user-specific protocols. Additionally, personalized formulas can be added for each user; the added formulas are first stored in the third storage unit 230 and then transmitted to the user device 110 and the HCP device 120 via the user interface module 600 or the HCP interface module 700.

Here, the third communication unit 210 facilitates data exchange between the NB Discovery module 200 and external systems. At the request of the third control unit 220, it transmits requests to the online research publication search engine 130 to periodically search for new publications, processes requests from the NB server 300, and transmits the most relevant NB protocols to other system components, such as the server, the user interface module 600, and the HCP interface module 700.

Furthermore, the third control unit 220 supervises the overall operation of the NB Discovery module 200, controls components, and coordinates data flow. It manages requests from external sources, such as the NB server 300, and supervises data retrieval from the online research publication search engine 130 to ensure that the system maintains up-to-date research results.

Additionally, the third storage unit 230 stores data processed or retrieved by the NB Discovery module 200, and the stored data includes personalized protocols, research results, and user-specific formulas. This third storage unit 230 can temporarily or permanently store data according to the needs of the system, and transmits requested data to the third control unit 220 or the third communication unit 210 when necessary.

Meanwhile, the NB Discovery module 200 serves to collect data from external sources such as new research announcements or user input. It further includes a data collection module 240 configured to work closely with the third communication unit 210 to collect relevant information, and an NB extraction module 250 configured to process the extracted protocols, convert the processed protocols into personalized formulas for the user, and generate relevant NBS scripts to convert the collected research data into runtime-generated executable LMAs evaluated within the LMS.

Additionally, when the NB Discovery module 200 transmits or receives data, the data exchange is managed by the third communication unit 210 under the control of the third control unit 220 or directly by the third control unit 220 itself.

Now, with reference to FIG. 5, the NB extraction module according to the present invention will be examined.

As illustrated in FIG. 5, the NB extraction module 250, as part of the NB Discovery module 200, processes unstructured clinical research papers written in natural language using a Large Language Model (hereinafter collectively referred to as “LLM”) and converts them into relevant Lifestyle Medicine guidelines. These guidelines are translated into a formalized, purpose-specific language called NBS to generate executable prescription parameters expressed in NBS.

This NB extraction module 250 includes a crawling module 251, an Optical Character Recognition (OCR) module 252, a sentence conversion module 253, an LM protocol extraction module 254, and an LMS conversion module 255.

Here, the crawling module 251 retrieves, via a network crawler and an LLM-based extraction component clinical research papers related to chronic diseases, and convert the identified and collected contents into text, tables, and images for further processing.

Additionally, for content provided in image format (e.g., scanned clinical papers), the OCR module 252 applies Optical Character Recognition (OCR) to extract text from such images and make it available for analysis.

Also, the sentence conversion module 253 rewrites the extracted text into a single, unified paragraph to facilitate modeling and analysis, and the rewritten text is then vectorized for Retrieval-Augmented Generation (RAG).

Furthermore, the LM protocol extraction module 254 serves to extract or import specific protocols of LM (e.g., guidelines for healthy lifestyle habits) from the system, and automatically extracts or presents customized LM protocols that fit the user's health status or lifestyle habits.

In addition, the LMS conversion module 255 converts the LMS protocol into a Nutribiometric Script.

Now, with reference to FIG. 6, the NB server according to the present invention will be examined.

The NB server 300 plays an important role in processing and organizing data related to LM prescriptions, converting the retrieved research results into NBS so that the system can generate actionable executable prescription parameters and LMAs. An example of this transformation is illustrated in FIG. 11, where the NB extraction module 250 automatically creates a database of guidelines related to clinical research, and the created database is structured in the NBS format. In this case, the user can input the current health status through the user interface module 600 of the user device 110. The system utilizes the LLM and a disease-related topic extraction module (not shown) to extract runtime-generated executable LMAs evaluated within the LMS for the user using the extracted disease-related topics, and converts them into NBS to allow for easy application and monitoring.

As shown in FIG. 6, the NB server 300 includes a fourth communication unit 310, a fourth control unit 320, a fourth storage unit 330, an NB prescription collection module 340, and an NB prescription analysis module 350.

Here, the fourth communication unit 310 facilitates communication between the NB server 300 and external devices such as the user device 110, HCP device 120, and NB Discovery module 200 through a communication network, thereby enabling real-time data exchange, which enables the latest lifestyle runtime-generated executable LMAs based on user-specific information.

Additionally, the fourth control unit 320 manages the overall operation of the NB server 300 and controls each component, processes requests from external systems, organizes collected data, and ensures that runtime-generated executable LMAs are appropriately converted into NBS format so that they can be used for prescriptions.

Also, the fourth storage unit 330 stores data related to lifestyle prescriptions, including research results and user-specific protocols, storing NB formulas and disease-specific topics so that they can be efficiently integrated into personalized runtime-generated executable LMAs.

Furthermore, the NB prescription collection module 340 is operated by the fourth control unit 320 and collects and processes data from various sources, such as the NB Discovery module 200, and collects input data, extracted text, processed data, and document conversions, organizing them into a structured directory.

In addition, the NB prescription analysis module 350 is operated by the fourth control unit 320, analyzes the directory organized by the NB prescription collection module 340, extracts relevant information such as disease-related topics, and organizes it in a file format, which is then used to create a personalized lifestyle prescription for the user.

Now, with reference to FIG. 7, the LMA Discovery module according to the present invention will be examined.

As illustrated in FIG. 7, the LMA Discovery module 400 according to the present invention serves to extract a list of Lifestyle Medicine Activities (LMAs) and to determine, for each extracted activity (e.g., consuming a specific food or running a mile), an activity-impact representation indicating how projected execution of the activity changes a patient's positional state within a Lifestyle Medicine Space (LMS). The extracted activities are converted into a formal, purpose-specific language called Lifestyle Medicine Activity Script (hereinafter collectively referred to as “LMAS”), wherein the LMAS encodes executable activity definitions and activity-to-LMS transformation logic configured to be executed by a runtime execution engine implemented by the NCP Engine within the LMS. For example, to achieve a goal provided by the NB Discovery module (e.g., 60-80 g of protein per day), when a user's current intake of protein is 50 g, the LMA Discovery module generates candidate runtime-generated executable LMAs defined in LMAS (e.g., “eat 2 boiled eggs” or “add 30 g of almonds”), and the runtime execution engine implemented by the NCP Engine executes the LMAS to project each candidate activity into the LMS and to compute resulting computational state transitions relative to the target constraint ranges.

This LMA Discovery module 400 includes a crawling module 401, an OCR module 402, a sentence conversion module 403, an LMA activity extraction module 404, and an LMS conversion module 405.

The crawling module 401 uses the LLM to retrieve chronic disease-related data and activity information in text form, and collects content in the form of text, tables, and images for further processing.

The OCR module 402 extracts text from an image format (e.g., a scanned document) through Optical Character Recognition (OCR) and converts it into analyzable data.

The sentence conversion module 403 rewrites the extracted text into cohesive paragraphs to facilitate analysis and modeling, and the transformed text is vectorized and utilized for RAG search and runtime-generated executable LMA generation.

The LMA activity extraction module 404 uses the LLM to extract specific activities (e.g., consuming a specific dish or performing an exercise) from the processed data and determine how such activities change the patient's position within the Lifestyle Medicine Space (LMS). This forms the basis for creating precise LM activity guidelines tailored to the needs of individual patients.

The LLM is used through individual AI Agents in the NB Discovery Module and the LMA Discovery Module. The LLM used in the AI Agent of the NB Discovery Module processes unstructured clinical research data written in natural language to extract the NBS formula that calculates each Lifestyle goal range. Meanwhile, the LLM used in the AI Agent of the LMA Discovery Module references unstructured [Lifestyle Activity name, impact on Lifestyle Activity goal range] data (e.g., “Eat 1 more The Protein”, “+20 g of protein”) to calculate an actionable ‘to-do list’ so that the user can reach the goal range. For example, to achieve the extracted target range (e.g., 60 g to 80 g of protein), a list of actionable activities (LMAs) (e.g., a list of Lifestyle Medicine Activities that include consuming one more ‘The Protein’) that allows the user to fall within the target range from the user's current position (e.g., consuming 50 g of protein) is calculated.

Finally, the LMS conversion module 405 converts extracted activity-impact data into LMAS as an executable representation, and the runtime execution engine implemented by the NCP Engine executes the LMAS to compute activity projections and state transitions in the LMS, thereby generating executable LMAs whose execution moves the user state toward the target constraint ranges derived from clinical research inputs.

Now, with reference to FIG. 8, the LMA server according to the present invention will be examined.

As illustrated in FIG. 8, the LMA server 500 according to the present invention includes an LMA search module 501, an LMA database 502, an LMA selection module 503, an LMA score processing module 504, and an LMA score display module 505.

Here, the LMA search module 501 quickly searches the LMA database 502 for the most semantically similar item when the user inputs an activity by name or description (e.g., “I want to eat a donut” or “I want to sleep for an hour”).

Additionally, the LMA database 502 is sophisticatedly designed to perform semantic searches, computing semantic similarity between an input activity representation and stored activity representations using an embedding-based retrieval function and matching it with the most relevant LMA. The LMA database 502 also includes additional information about, for example, typical serving sizes of donuts, alternative serving sizes, or variations in activities, so that the user can easily identify different types of donuts (see FIG. 12).

Furthermore, when the LMA search module 501 finds a relevant LMA, the LMA selection module 503 displays attractive options to the user based on the finding. These options include details such as serving size, time duration (e.g., for activities such as sleep or exercise), or various categories (e.g., meditation types), allowing the user to select an attractive LMA and specify the amount, duration, or type according to the displayed options.

Also, after the user selects a desired LMA, the LMA score processing module 504 precisely calculates the degree to which the corresponding activity contributes to the user's daily multidimensional score. These scores represent the impact of the selected activity on the user's overall lifestyle adherence, including its impact on dimensions such as nutrition, exercise, or stress management.

Additionally, the LMA score display module 505 displays the user's daily multidimensional score calculated by the LMA score processing module 504, scores the results of LM-related activities or health assessments, and visually displays the scores. For example, after evaluating the user's exercise, eating habits, stress level, and the like, this is converted into a score and displayed to the user. The user's current score is displayed relative to the “Green Zone” indicating the level of adherence optimized for the personalized LM prescription.

Now, the NCP engine according to the present invention will be examined with reference to FIG. 9.

As illustrated in FIG. 9, the NCP engine 900 according to the present invention generates a personalized LMA list based on user-specific data, user LMA history, and biometric history.

The runtime execution engine implemented by the NCP engine 900 interprets and executes executable domain-specific scripts (e.g., NBS and LMAS) to dynamically define evaluation logic and state-transition behavior at runtime, such that modification of system behavior is achieved through script execution rather than modification of underlying application code or predefined schema definitions. By integrating various modules including an NCP bot 910 that generates daily tasks (LMAs, surveys, educational materials, and coaching messages) based on instructions obtained from the LMA server 500 and the NCP design server 930, it provides real-time updates and support via the user device 110 and the HCP device 120 so that user behavior aligns with the personalized health plan.

The NCP engine 900 according to the present invention includes an NCP bot 910, an NCP design server 930, an LMA selection module 950, and a biometric input module 970.

Here, the NCP bot 910 is connected to the NCP design server 930, the LMA selection module 950, and the biometric input module 970 to control these modules and manage the overall operation of the NCP engine 900.

Additionally, the NCP design server 930 stores various NCPs and allows searching by name or description (e.g., “4-Week Weight Management for Healthy Men” or “Post-Surgery Recovery”).

Upon request from the NCP bot 910, the NCP design server 930 provides the LMA and survey for each date of the program.

Additionally, the LMA selection module 950 supports users in freely selecting meals and recording their intake, or selecting pre-determined meal options, and allows them to preview food products before making a final selection.

Furthermore, the biometric input module 970 is configured to record the patient's biometric data, allowing the patient and physician to monitor the trend of LMA intake and resulting biometric changes, and to evaluate the patient's progress.

Meanwhile, the NCP bot 910 calculates each user's daily to-do list and displays it on the user device 110. The displayed list includes LMAs, surveys, educational materials, and health record management tasks, facilitating systematic remote management. The NCP bot 910 also includes a date module 911 that records user responses such as completed or replaced LMAs, manages time-related tasks, and stores and processes data on important dates and events in the user's activity history. It also stores data in a plurality of user databases 912 to 917.

The plurality of user databases 912 to 917 include: a user InBody DB 912 for storing body composition data and biometric data; a user NCP history DB 913 for recording the user's nutritional management process history; an educational material DB 914 for recording educational materials; an InBody data input DB 915 for recording data measured by biometric equipment; a survey input DB 916 for recording survey data; and a nutrition DB 917 for recording food information, nutritional ingredients, food quantities, and sources of nutritional information. The plurality of user databases 912 to 917 store and manage relevant user data, which is analyzed in real time to calculate additional LMAs for reaching the Green Zone.

Meanwhile, when the user device 110 reports that a daily action (based on the 6 Pillars of LM) is completed, the system sends a notification to the HCP device 120. The NCP bot 910 uses the date module 911 to check the current date and transmits necessary monitoring data, such as user information, display date, and expiration date, to the HCP device 120, while the NCP design server 930 provides NCP settings and daily action definitions.

Additionally, the NCP engine 900 retrieves relevant user and prescription data daily to ensure that correct user-activities and runtime-generated executable LMAs are displayed on the user device 110, and the NCP design server 930 standardizes and customizes all aspects of the nutrition management process according to the user's personal health needs.

Now, the NCP simulator according to the present invention will be examined with reference to FIG. 10.

As illustrated in FIG. 10, the NCP simulator 800 according to the present invention serves to calculate a list of LMAs that ensures the user's overall LM score remains within the Green Zone, defined as an acceptable range for each lifestyle prescription component (e.g., sodium intake<2000 mg, alcohol consumption<1 g). In certain embodiments, the NCP simulator 800 operates as a computational sub-module invoked by the runtime execution engine implemented by the NCP engine 900, and performs simulation and candidate evaluation functions under control of the NCP engine rather than independently determining evaluation logic. Each LMA contributes to the user's overall LM score by considering its relation to food, exercise, or sleep.

This NCP simulator 800 includes an LMA score calculation module 801, a context fine-tuning module 802, and an additional LMA generation module 803.

Here, the LMA score calculation module 801 calculates minimum and maximum scores for each component of the user's prescription. This helps define the acceptable range for each activity and ensures that the user's choices remain within safe limits.

Additionally, the context fine-tuning module 802 plays a role in dynamically adjusting the details of the nutrition management process by reflecting the user's personal context in the NCP process.

The user can provide specific context or preferences to guide the NCP simulator 800. For example, a user may state, “The total calorie Green Zone forces me to eat too little, so I will eat more and burn the excess calories through exercise.” In this case, the context fine-tuning module 802 computationally selects, from candidate runtime-generated executable LMAs, an runtime-generated executable LMA whose projected state transition in the LMS satisfies the target constraint ranges while meeting the user-provided context constraints. This allows prescribed activities to be tailored to the user's goals and lifestyle choices.

Additionally, the additional LMA generation module 803 builds a collection of LMAs whose scores aggregate within the Green Zone according to the user's preferences and context. This additional LMA generation module 803 carefully selects activities that satisfy the user's individual preferences while ensuring that the overall LM score remains within an acceptable range.

Meanwhile, the context fine-tuning module 802 displays a list of LMAs generated by the system and is connected to the LMA selection module 503, which allows the user to review activities and check details on how each activity contributes to their overall score and lifestyle prescription adherence.

Now, with reference to FIG. 11, an example of the NB Discovery module converting an online clinical research publication into an NBS according to the present invention will be examined.

As illustrated in FIG. 11, the example where the NB Discovery module converts an online clinical research publication into an NBS shows the automatic creation of a database of guidelines related to clinical research, and the created database is structured in the NBS format.

Here, the crawling module 251 collects images for various clinical studies targeting patients with chronic diseases such as obesity. The OCR module 252 automatically checks whether additional information can be extracted from the collected images and applies it (an example output of the crawling module 251 and OCR module 252 is “A new predictive equation for resting energy expenditure in healthy individuals” by Mark D. Mifflin). The sentence conversion module 253 parses the document into sentences. Each analyzed sentence is reviewed by the Lifestyle Medicine protocol extraction module 254 to extract formulas for Resting Energy Expenditure (REE), such as “REE=10*weight+6.25*height−5*age+5” for men and “REE=10*weight+6.25*height−5*age−161” for women. The LMS conversion module 255 converts these extracted formulas into a Lifestyle Medicine Script expression, for example, as follows: calories=Switch($[sex]==1=>10*$[weight]+6.25*$[height]−5*$[age]+5 true=>10*$[weight]+6.25*$[height]−5*$[age]−161)

In the above expression, the definitions of the Asterisk (*) and other marks are as follows.

### 1. Asterisk (*):

Role: Represents multiplication.

Example: “10*$[weight]” means the result of multiplying the weight value ($[weight]) entered by the user by 10.

2. $[ ], #[ ] sign marks ($ and #):
$[ ]: Indicates a variable that requires additional input.
Example: $[weight_kg] refers to weight data that the user must directly input.
#[name]: References a value that already exists in the database.
Example: #[date-of-birth] refers to birth date data stored in the database.

### 3. Switch Function:

Role: Applies different formulas depending on the condition.
Principle of Operation:
If the condition “$[sex]==1” is true, the first formula is applied: “10*$[weight]+6.25*$[height]−5*$[age]+5”.
If the condition is false, the second formula is applied: “10*$[weight]+6.25*$[height]−5*$[age]−161”

### Example

If the gender is male ($[sex]==1): “10*$[weight]+6.25*$[height]−5*$[age]+5” calculates calories.

If the gender is female ($[sex]!=1): “10*$[weight]+6.25*$[height]−5*$[age]−161” calculates calories.

This implements a method of dynamically calculating calories according to gender and user input.

### 4. Method of Indicating Tolerance Range:

The Green Zone visualizes and displays a tolerance range instead of a single specific value.

Therefore, in NBS, it is common to indicate a tolerance range rather than specifying an exact value.

For example, if the average daily calories are 1400 kcal and a margin of error of ±10% is allowed, the NBS script expresses the allowable range as follows: calories=1400*0.9~1400*1.1; The following are additional examples:
“Consume at least 0.5 g of omega-3 daily” is expressed in NBS script as omega-3=0.5~;
“There is no specific limit on alcohol” is expressed as alcohol=~;
Now, an example of the user interface of the user device will be examined with reference to FIGS. 12A and 12B.

As illustrated in FIGS. 12A and 12B, the example of the user interface of the user device 110 shows visualization of executable constraint ranges (Green Zone) and computed adherence states produced by executing NBS as part of an intermediate executable representation processed by the runtime execution engine implemented by the NCP Engine. The prescription given as an example is automatically converted to a numerical value.

On the right side of FIG. 12A, a personalized numerical prescription is displayed based on the patient's body measurement data (e.g., height, weight, age), and on the left side of FIG. 12A, the numerical range on the right is visualized as a green horizontal bar. Here, the dark gray notation represents the patient's overall position within various components of the Lifestyle Space.

In the examples of FIGS. 12A and 12B, the Lifestyle Space (LMS) consists of items such as daily calorie intake, carbohydrates, sugars, fat, cholesterol, saturated fat, omega-3, omega-3 to omega-6 ratio, protein, sodium, and alcohol. Additionally, in this example, the patient is asked to perform additional Lifestyle Medicine Activities (LMAs) to increase their intake of calories, carbohydrates, fats, omega-3s, and protein.

For example, referring to the left side of FIG. 12B, assuming that the user consumed one donut, the runtime execution engine implemented by the NCP Engine computes that the projected activity places the user state outside the target constraint ranges (Green Zone) in the LMS. On the other hand, on the right side of FIG. 12B, the runtime execution engine implemented by the NCP Engine computes that when the user consumes ⅔ of a donut, the projected activity places the user state within the executable constraint ranges (Green Zone) in the LMS. These examples demonstrate the utility of the system in visually tracking and managing lifestyle medicine prescription adherence through NBS.

Now, an example showing the NCP definition will be examined with reference to FIG. 13.

As illustrated in FIG. 13, FIG. 13 shows an example of a Nutritional Care Process (NCP) that provides a list of LMAs designed to adhere to a lifestyle medicine prescription. In the example of FIG. 13, as long as the patient follows the dynamically generated and evaluated LMAs in this list, the patient can adhere to the given lifestyle medicine prescription because the LMAs are designed to meet the patient's daily lifestyle medicine prescription. This prescription consists of a 28-day plan.

On the first day of the NCP, the NCP engine generates a set of runtime-generated executable LMAs specifying consumption of half a bagel, a plate of cucumber salad, and half a banana for breakfast, such that execution of the LMAs projects the user state toward the target constraint ranges (Green Zone) in the LMS. Whenever a patient practices the breakfast items on the first day, they can check their prescription compliance status using FIG. 12. Surveys and educational materials may also be added as part of the LMA.

Now, an example displayed on the user device will be examined with reference to FIG. 14.

As shown in FIG. 14, it shows how information is displayed to the patient through the user device.

During the patient's NCP period, a daily to-do list is automatically generated according to the NCP design. The to-do list items may include various types, such as dietary items like breakfast, lunch, dinner, and snacks; daily recording of InBody measurements; periodic surveys and educational materials; to-do lists related to physical and social activities; and items such as medication if necessary.

For example, an example displayed on a user device is as follows:

### 1. Meal Record Summary:

Breakfast: Recorded as “Seasoned Cashews and 3 others”, indicating that there are additional items consumed.
Lunch: “Medisola: Nutritional Balance Salmon Steak” is displayed, showing the record of the menu consumed for lunch.
Dinner: Displayed as “Kimbap and 1 other”, indicating that there are additional consumed items.
Snacks: Recorded as “Tomatoes and 2 others”, including snack intake history.

### 2. Survey:

V Questionnaire: The “Korean Mediterranean Diet Measurement” item is displayed, and survey data can be modified or entered via the pencil icon on the right.
The eye-shaped icon on the right represents a button that allows checking the currently entered data.

### 3. Health Information:

Weight Measurement: Weight measurement data is recorded and can be modified via the pencil icon.
The eye-shaped icon on the right allows checking the currently entered health information.

Now, an example of an LMA detailed list displayed on the user device will be examined with reference to FIG. 15.

As illustrated in FIG. 15, the example of the LMA detailed list displayed on the user device is a screen that visually provides detailed information on breakfast and nutritional information according to the intake amount. This screen analyzes the intake status of major nutrients based on the meal data recorded by the user and displays it by comparing it with the target constraint ranges (Green Zone) computed by executing the NBS as part of an intermediate executable representation processed by the runtime execution engine implemented by the NCP Engine.

### 1. Food Selection and Intake Input:

For example, “Seasoned Cashews”, “Plain Yogurt”, “Roasted Walnuts”, and “Americano” are selected, and the intake amount can be adjusted via the drop-down menu next to each item.
For example, “Seasoned Cashews” is set to 10 pieces.

### 2. Breakfast Intake Chart:

At the bottom, a chart visualizing the nutrient intake information of the breakfast entered by the user is displayed.
Nutrient List: Includes calories, carbohydrates, total sugars, total dietary fiber, fat, cholesterol, saturated fatty acids, trans fatty acids, omega-3, and omega-3:omega-6 ratio.
Comparison with Target Range:
   For each item, if the intake is within the target range, it is displayed in green, and if it is exceeded or insufficient, it is highlighted in a different color (e.g., yellow).
   For example, the intake of “Omega-3” (0.9 g) is slightly below the target range and is displayed in yellow.

Now, an example of NBS according to the present invention will be examined with reference to FIG. 16.

As illustrated in FIG. 16, an example of an NBS according to the present invention is as follows.
1. Defines the acceptable daily intake range for Omega-3.
2. Uses the Switch function to calculate different Omega-3 targets based on the user's gender.
3. If the user's gender is male (“Male”), the target is set to 2 g; otherwise, it is set to 1.5 g.
4. The allowable range specifies only the starting value (minimum value), and the upper limit is not specified separately.

Overall, NBS is designed to dynamically calculate customized target values for each nutrient based on the user's body information and input data, and set acceptable ranges for those targets, thereby evaluating whether they are within the “Green Zone” by comparing them to actual intake. For example, regarding calories, if the user's goal is calculated as 2000 kcal, a range of 1800 kcal (2000×0.9) to 2200 kcal (2000×1.1) is allowed, and if the user consumes within this range, it can be determined that the goal has been achieved.

Additionally, each expression within the NBS script is configured to precisely calculate and display personalized runtime-generated executable LMAs by utilizing functions, variables, conditional statements (Switch), range symbols (~), and the like.

Accordingly, for example, the prescription “Maintain 60-80 g of protein daily” is mathematically expressed by NBS as “protein=60~80;”.

As described above, the personalized real-time action-based healthcare system adaptable to any prescription according to the present invention uses NBS to compute dynamically adjusted user-specific executable constraint ranges and to generate executable LMAs through an intuitive application and web interface, wherein the executable constraint ranges form part of an intermediate executable representation processed by a runtime execution engine implemented by the NCP Engine. By guiding patients to adhere to the LMA, it improves upon the limited and rigid approach of existing fixed prescriptions and supports patients in achieving their health goals in a more flexible and personalized manner, thereby improving long-term health outcomes. In addition, by automatically reconstructing the LMS, which is a multidimensional space defined by the 6 Pillars, based on the description provided by NBS, there is no need for manual adjustment or system migration when the LMA changes according to changes in the patient's health status.

As described above, the present invention has been described based on preferred embodiments, but these embodiments are intended to illustrate rather than limit the present invention. Therefore, those skilled in the art will appreciate that various changes, modifications, or adjustments to the above embodiments are possible without departing from the technical spirit of the present invention. Therefore, the scope of protection of the present invention should be interpreted to include all examples of changes, modifications, or adjustments that fall within the gist of the technical idea of the present invention.

[REFERENCE NUMERALS]

| | |
|---|---|
| 110: User Device | 111: First Input Unit |
| 113: First Display Unit | 115: First Communication Unit |
| 117: First Storage Unit | 119: First Control Unit |
| 120: HCP Device | 121: Second Input Unit |
| 123: Second Display Unit | 125: Second Communication Unit |
| 127: Second Storage Unit | 129: Second Control Unit |
| 130: Online Research | 200: NB Discovery Module |
| Publication Search Engine | |
| 210: Third Communication Unit | 220: Third Control Unit |
| 230: Third Storage Unit | 240: Data Collection Module |
| 250: NB Extraction Module | 251, 401: Crawling Module |
| 252, 402: OCR Module | 253, 403: Sentence Conversion |
| | Module |
| 254: LM Protocol | 255, 405: LMS Conversion Module |
| Extraction Module | |
| 300: NB Server | 310: Fourth Communication Unit |
| 320: Fourth Control Unit | 330: Fourth Storage Unit |
| 340: NB Prescription | 350: NB Prescription Analysis |
| Collection Module | Module |
| 400: LMA discovery module | 404: LM Activity Extraction Module |
| 500: LMA Server | 501: LMA Search Module |
| 502: LMA Database | 503: LMA Selection Module |
| 504: LMA Score | 505: LMA Score Display Module |
| Processing Module | |
| 600: User Interface Module | 700: HCP Interface Module |
| 800: NCP Simulator | 801: LMA Score Calculation Module |
| 802: Context | 803: Additional LMA |
| Fine-Tuning Module | Generation Module |
| 900: NCP Engine | 910: NCP Bot |
| 911: Date Module | 912: User InBody DB |
| 913: User NCP History DB | 914: Educational Materials DB |
| 915: InBody Data Input DB | 916: Survey Input DB |
| 917: Nutrition DB | 930: NCP Design Server |
| 950: LMA Selection Module | 970: Biometric Input Module |

1. A personalized real-time action-based healthcare system adaptable to any prescription, comprising:
a user device configured to input user biometric measurements and user-specific data;
a healthcare professional (HCP) device connected to the user device and configured for a healthcare professional to review, monitor, and adjust a prescription;
an online research publication search engine configured to access clinical research publications via an online search engine;
an NB (NutriBiometric) Discovery module configured to interpret a Lifestyle Medicine (LM) prescription provided by the user device or the HCP device, convert the prescription into the Nutribiometric Script (NBS), and derive lifestyle goals for chronic diseases, nutrients, and exercise; and
an LMA (Lifestyle Medicine Activity) Discovery module configured to generate candidate executable activity scripts (LMAS) defining activity-to-LMS transformation logic and executable constraints, for execution by the runtime execution engine to produce runtime-evaluated LMAs as state-transition operator instances,
wherein the prescription is encoded as a Nutribiometric Script (NBS) comprising an executable domain-specific script, the NBS defining executable evaluation logic and one or more evaluation dimensions, and wherein a runtime execution engine implemented by an NCP Engine executes the NBS or an intermediate executable representation compiled therefrom to dynamically instantiate, at runtime, a Lifestyle Medicine Space (LMS) as a computational evaluation environment wherein the runtime execution engine establishes a prescription-defined virtual execution substrate whose computational topology, including evaluation dimensions, dependency relationships, constraint evaluators, and state vector structure, is deterministically generated from executable script semantics at runtime rather than predefined within application code or database schema, in which the evaluation dimensions, machine-readable evaluation variables, and constraint relationships are synthesized at runtime without reliance on predefined measurement categories, fixed schemas, or static ontologies wherein evaluation dimensionality, dependency relationships, and computational state vector structure are derived from executable script semantics and instantiated as structural elements of the computational evaluation environment rather than mapped onto pre-existing application data models or database schemas, and wherein execution of the NBS causes the system to deterministically generate and update computational state representations within the LMS and to perform automated evaluation of user activities by computing computational state transitions that update a user's current LMS position relative to target constraint ranges.

2. The system of claim 1,
further comprising an NB server configured to process data received from the NB Discovery module to compute, by machine execution of the domain-specific script, specific observable and measurable personalized user goals as target constraints within a Lifestyle Medicine Space (LMS) associated with the six pillars of Lifestyle Medicine, the NB server defining computational evaluation dimensions dynamically based on the domain-specific script and corresponding target ranges within the LMS, and being further configured to provide structured goal definitions enabling tracking of lifestyle prescription adherence through evaluation of lifestyle activities relative to the target ranges.

3. The system of claim 2,
further comprising an LMA (Lifestyle Medicine Activity) Discovery module configured to analyze and generate proposed executable lifestyle medicine activities (LMAs) based on a user's current health status, prescription, and input data, and to evaluate, via projection into the Lifestyle Medicine Space (LMS), each generated user-tailored lifestyle action comprising specific dietary adjustments or physical activities in accordance with the prescription processed by the NB server by invoking, using machine execution of the domain-specific script, an activity-to-LMS transformation executed by the NCP engine using goal definitions generated by the NB server, the transformation projecting each activity into a Lifestyle Medicine Space (LMS) associated with the six pillars of Lifestyle Medicine to determine impact relative to personalized user goals.

4. The system of claim 3,
further comprising an LMA server configured to cooperate with the LMA Discovery module to store and manage the LMA, track the user's progress, monitor completed activities, and determine, based on projection results of lifestyle activities into the Lifestyle Medicine Space (LMS) received from the NCP engine, whether the user's daily life activities fall within a Green Zone representing a target range for the user's LM prescription.

5. The system of claim 4, further comprising
an NCP (Nutritional Care Process) simulator configured to execute state transition simulations within the LMS to compute positional state transitions representing LM compliance by simulating, via machine execution of the domain-specific script that defines executable evaluation logic and dynamically constructs evaluation dimensions within the LMS, contributions of one or more LMAs to changes in a user's current position within the Lifestyle Medicine Space (LMS), wherein user behavior represented as a set of LMAs produces positional changes within the LMS, and by computationally updating LMS state based on user activities within the Lifestyle Medicine Space (LMS) corresponding to the six pillars of Lifestyle Medicine based on inputs from the LMA Discovery module and the NB server.

6. The system of claim 5, further comprising
an NCP engine configured to operate as a runtime execution engine implemented by the NCP Engine that executes the executable domain-specific script to instantiate and update the Lifestyle Medicine Space (LMS), and to calculate executable personalized health goals as LMS target constraints for each user and dynamically adjust the goals in real time based on user input, changing prescriptions, or evolving health conditions, wherein the NCP engine communicates directly with the LMA Discovery module and the LMA server to continuously improve the user's LMA.

7. The system of claim 6, wherein
the NB Discovery module
searches unstructured clinical research papers written in natural language using a Large Language Model (LLM), extracts formulas from the searched clinical research papers, integrates the formulas into user-specific protocols to add personalized formulas for each user, extracts relevant protocols required for Lifestyle Medicine interventions, and converts the extracted protocols into Nutribiometric Scripts (NBS) defining evaluation logic within the Lifestyle Medicine Space (LMS) for nutritional protocols and other LMAs.

8. The system of claim 7, wherein
the NB Discovery module further comprises:
a data collection module configured to collect data from external sources; and
an NB extraction module configured to generate executable NBS scripts defining evaluation logic within the Lifestyle Medicine Space (LMS) based on outputs of the LLM extraction of claim 7 from the data collected by the data collection module.

9. The system of claim 8, wherein
the NB server comprises:
an NB prescription collection module configured to collect and process data input from the NB Discovery module, and collect input data, extracted text, processed data, and document conversions to compile them into executable data structures supporting domain-specific script execution within the Lifestyle Medicine Space (LMS); and
an NB prescription analysis module configured to analyze the directory organized by the NB prescription collection module to extract relevant information on disease-related topics and organize the information into a file format.

10. The system of claim 4, wherein
the LMA Discovery module extracts relevant LMAs from unstructured clinical research papers written in natural language using an LLM, and converts the extracted activities into a purpose-specific language called Lifestyle Medicine Activity Script (LMAS) defining activity-to-LMS transformation logic executable within the Lifestyle Medicine Space (LMS), thereby enabling executable activity definitions evaluated within the LMS.

11. The system of claim 10, wherein
the LMA server comprises:
an LMA search module configured to search for a most semantically similar item in an LMA database when the user's activity is input by name or description by the user device;
the LMA database sophisticatedly designed to perform semantic searches, deeply understand user input, and match the input with a most relevant LMA;
an LMA selection module configured to display options to the user based on the relevant LMA found by the LMA search module;
an LMA score processing module configured to precisely calculate, using executable LMS evaluation logic generated from the domain-specific script, a degree to which a corresponding activity produces positional change within the Lifestyle Medicine Space (LMS)
after the user selects a desired LMA; and
an LMA score display module configured to display the user's daily multidimensional score calculated by the LMA score processing module, score results of LM-related activities or health assessments, and visually display the scores.

12. The system of claim 5, wherein
the NCP simulator comprises:
an LMA score calculation module configured to compute target constraint ranges corresponding to evaluation dimensions within the Lifestyle Medicine Space (LMS);
a context fine-tuning module configured to incorporate user-specific context into simulation parameters without altering executable evaluation logic defined by the domain-specific script; and
an additional LMA generation module configured to generate candidate LMAs whose projected execution, determined via machine execution of the domain-specific script, produces state transitions within the LMS that maintain or move a user position toward target constraint ranges associated with the six pillars of Lifestyle Medicine.

13. The system of claim 6, wherein
the NCP engine comprises an NCP bot, an NCP design server, an LMA selection module, and a biometric input module,
wherein the NCP design server stores predefined NCP definitions associated with executable domain-specific scripts defining LMS evaluation logic;
wherein the biometric input module records user biometric data used to update a current positional state of the user within the Lifestyle Medicine Space (LMS);
wherein the NCP bot is configured to dynamically generate and update executable lifestyle medicine activities (LMAs), surveys, and task definitions based on computational evaluation of LMS state transitions produced via machine execution of the domain-specific script; and
wherein the NCP bot communicates with the LMA Discovery module and the LMA server to continuously refine LMAs based on changes in the user's LMS position relative to personalized target constraints.

14. The system of claim 1, wherein the executable domain-specific script defines one or more evaluation dimensions not preconfigured in a system schema, and wherein the instantiated computational evaluation space is generated at runtime based on the script-defined evaluation dimensions.

15. The system of claim 1, wherein the NB Discovery module defines evaluation variables and constraint relationships forming the instantiated computational evaluation space, and wherein the LMA Discovery module generates candidate lifestyle actions by operating within the instantiated computational evaluation space without modifying the evaluation variables or constraint relationships.

16. The system of claim 1, wherein the executable domain-specific script is compiled into an intermediate executable representation comprising:
a symbol table defining evaluation dimensions,
executable constraint ranges representing personalized targets, and
executable evaluators,
and wherein a runtime execution engine implemented by the NCP Engine executes the intermediate executable representation to instantiate a multidimensional computational evaluation environment in which evaluation dimensions are dynamically synthesized at runtime without reliance on predefined schemas.
